(19) United States
(12) Patent Application Publication
(10) Pub. No.: US 2008/0254043 A1
(43) Pub. Date: Oct. 16, 2008

(54) Drug For Treating Ischemic Disease

(75) Inventors: Kazuo Umemura, Shizuoka (JP); Atsushi Muto, Shizuoka (JP); Sachiya Ikeda, Shizuoka (JP)

Correspondence Address: FOLEY AND LARDNER LLP;SUITE 500, 3000 K STREET NW, WASHINGTON, DC, 20007, US

(73) Assignee: Chugai Seiyaku kabushiki Kaisha National University Corporation Hamamatsu

(21) Appl. No.: 11/883,646

(22) PCT Filed: Feb. 7, 2006
(86) PCT No.: PCT/JP2006/302022

§ 371 Date: Apr. 7, 2008

(30) Foreign Application Priority Data
Feb. 7, 2005 (JP) 2005-030836

(51) Int. Cl. A61K 39/395; A61P 9/10

(52) U.S. Cl. 424/172.1

(57) ABSTRACT

The present invention relates to a pharmaceutical composition which includes a tissue factor inhibitor, which is administered after the onset of a thromboembolic ischemic disease, particularly after thrombus or embolus formation; a secondary thrombus formation inhibitor which includes a tissue factor inhibitor, which is administered after primary thrombus formation; a blood flow reduction inhibitor which includes a tissue factor inhibitor to be administered after thrombus formation or embolism formation; and a method of suppressing secondary thrombus formation by inhibiting tissue factor function following formation of a primary thrombus.

### BACKGROUND ART

The present application relates to a pharmaceutical composition which includes a tissue factor inhibitor to be administered following the onset of thromboembolic ischemic disease. The invention also relates to a pharmaceutical composition which includes a tissue factor inhibitor and which is used for inhibiting secondary thrombus formation and/or for preventing blood flow reduction.

In a blood vessel, when an abnormality in the ability to regulate coagulation and fibrinolysis occurs by various factors, occlusion of the blood vessel by a formed thrombus may cause circulatory failure (thrombosis or thromboembolism), triggering in turn ischemic disease and possibly leading to a lethal condition.

Such thrombosis or thromboembolism is classified as venous thrombosis or arterial thrombosis, depending on where the vascular occlusion occurs. Venous thromboses include deep-vein thrombosis of the lower limbs, pulmonary embolism and cerebral sinus thrombosis. Arterial thromboses include brain infarction and myocardial infarction. Ischemia due to thrombus formation also occurs in, for example, transient ischemic attack (abbreviated below as “TIA”), angina pectoris and chronic arterial obstruction.

In ischemic disease arising from thrombosis or thromboembolism, sometimes the ischemic tissue incurs irreversible damage due in part to a decrease in the oxygen feed rate caused by the reduction in blood flow, as a result of which severe symptoms accompanied by sequelae and complications are exhibited. On the other hand, if the impairment in blood perfusion is removed and blood flow resumes within a short time following onset, the ischemic tissue may recover without incurring damage. Therefore, in the treatment of such ischemic disease, appropriate treatment at the acute stage, especially shortly after onset, is important for holding the reduction in blood flow to a minimum. Drug therapy currently used in these diseases includes antiplatelet drugs such as aspirin, thrombolytic drugs such as urokinase, and anticoagulants such as warfarin and heparin. However, these drugs are often used primarily to avoid complications and prevent a recurrence. At present, drug therapies which exhibit adequate effects at the acute and hyperacute stages have yet to be established.

Tissue plasminogen activators (t-PA), which are regarded as effective for treating brain infarction, are limited to use within 3 hours of onset due to the side effects of intracerebral hemorrhaging. Hence, in the clinical setting, there exists a need for a drug having a wide therapeutic time window (TTW), particularly when administered following onset.

Tissue factor (abbreviated below as “TF”) is a 30 kDa transmembrane-type glycoprotein which expresses on the surface of cells. As the receptor for blood coagulation factor VII, it functions as the substantial initiation factor for the blood coagulation reaction (see Non-Patent Document 1: Thromb. Haemost. Volume 74(1), page 180-184 (1995)). TF forms a complex with blood coagulation factor VII, thereby activating blood coagulation factors IX and X. In a normal state, TF is substantially absent from the vascular endothelium and tissue directly exposed to the blood (blood cells, etc.). However, TF is abundant in the vascular adventitia and connective tissue, and functions to minimize bleeding by activating the blood coagulation system when vascular injury has occurred.

In recent research, anti-TF antibodies and other substances which inhibit the blood coagulation action of TF are used as safe and effective anticoagulants with few side-effects such as bleeding. Humanized anti-TF antibodies in particular are believed to have the potential of becoming excellent treating agents which are safe, effective and have a long blood retention time (see Patent Documents 1 to 8).

For example, human/mouse chimeric antibodies composed of the variable region (V region) from a mouse monoclonal antibody to human TF and the constant region (C region) from a human antibody, and humanized antibodies in which the complementarity determining regions of the light chain (L chain) V region and heavy chain (H chain) V region of a mouse monoclonal antibody to human TF have been grafted to a human antibody, have been reported to show promise as excellent therapeutic agents for disseminated intravascular coagulation (DIC), arterial thromboses and venous thromboses (Patent Document 7: WO 99/51743; Patent Document 8: WO 01/24626).

Research is also being carried out on the therapeutic effects by substances which inhibit the blood coagulation action of TF on thrombotic or thromboembolic ischemic disease. For example, the amount of thrombus formation and the blood flow at the time of blood reperfusion following occlusion of the middle cerebral artery (abbreviated as “MCA” below) for a given length of time in experimental animals (baboons) administered anti-human TF monoclonal antibodies prior to the onset of vascular occlusion have been reported (Non-Patent Document 2: Stroke volume 25, page 1847-1854 (1994); Non-Patent Document 3: Stroke volume 24, page 847-854 (1993)). However, the therapeutic efficacy of substances which inhibit the blood coagulation action of TF when administered following the onset of vascular occlusion has not been reported.
Patent Document 1: International Publication WO 88/07543 Pamphlet
Patent Document 2: International Publication WO 96/40921 Pamphlet
Patent Document 3: International Publication WO 98/40408 Pamphlet
Patent Document 4: International Publication WO 01/70984 Pamphlet
Patent Document 5: International Publication WO 03/37911 Pamphlet
Patent Document 6: International Publication WO 03/93422 Pamphlet
Patent Document 7: International Publication WO 99/51743 Pamphlet
Patent Document 8: International Publication WO 01/24626 Pamphlet
Non-Patent Document 1: Thromb. Haemost. Volume 74(1), 180-184 (1995)
Non-Patent Document 2: Stroke volume 25, page 1847-1854 (1994)
Non-Patent Document 3: Stroke volume 24, page 847-854 (1993)

### DISCLOSURE OF THE INVENTION

### PROBLEMS TO BE SOLVED BY THE INVENTION

An object of the invention is to provide a pharmaceutical composition capable of exhibiting a therapeutic effect when administered following the onset of thrombotic or thromboembolic ischemic disease. Another object of the invention is to provide a drug treatment means which may be used following the onset of thromboembolic ischemic disease.

### MEASURE FOR SOLVING THE PROBLEMS

The inventors have conducted extensive research in order to resolve the above problems. As a result, they have discovered that therapeutic effects against such ischemic disease can be obtained by administering a TF inhibitor following the onset of thromboembolic ischemic disease, and have achieved the present invention.

According to one aspect of the invention, there is provided a pharmaceutical composition comprising a TF factor inhibitor, which is administered following onset of thromboembolic ischemic disease. The pharmaceutical composition may be used to treat, for example, thromboembolic ischemic disease. The thromboembolic ischemic disease may be, an infarction such as a brain infarction. The tissue factor inhibitor may be, for example, an antibody which binds to tissue factor.

According to another aspect of the invention, there is provided a pharmaceutical composition comprising a tissue factor inhibitor, which is administered following thrombus or embolism formation. The pharmaceutical composition may be used to treat, for example, thromboembolic ischemic disease. The thromboembolic ischemic disease may be an infarction such as a brain infarction. The tissue factor inhibitor may be, for example an antibody which binds to tissue factor.

According to yet another aspect of the invention, there is provided a secondary thrombus formation preventing agent comprising a tissue factor inhibitor, which is administered following primary thrombus formation. Here, the tissue factor inhibitor may be an antibody which binds to tissue factor.

According to a further aspect of the invention, there is provided a blood flow reduction preventing agent comprising a tissue factor inhibitor, which is administered following thrombus or embolism formation. Here, the tissue factor inhibitor may be an antibody which binds to tissue factor.

According to a still further aspect of the invention, there is provided a method for suppressing secondary thrombus formation, which includes inhibiting tissue factor function following formation of a primary thrombus.

According to an additional aspect of the invention, there is provided a method for suppressing enlargement of an infarction, which includes inhibiting tissue factor function following onset of the infarction.

According to yet another aspect of the invention, there is provided a method for suppressing a reduction in blood flow, which includes inhibiting tissue factor function following thrombus formation or embolism formation.

### EFFECT OF THE INVENTION

The present invention provides a pharmaceutical composition which can be used following the onset of thrombotic or thromboembolic ischemic disease. Administration of the pharmaceutical composition of the present invention is useful as a measure taken in drug therapy following the onset of such disease.

### BRIEF DESCRIPTION OF THE DRAWINGS

FIG. 1 shows, in a mouse MCA occlusion model by the PIT method, the brain infarction volume ratios for groups administered 0.1 mg/kg or 1 mg/kg of anti-human TF antibody immediately after the end of light exposure (Graphs A, B and C).

FIG. 2 shows, in a mouse MCA occlusion model by the PIT method, the brain infarction volume ratios for groups administered 0.01 mg/kg, 0.03 mg/kg or 0.1 mg/kg of anti-human TF antibody immediately after the end of light exposure (Graphs A, B and C).

FIG. 3 shows, in a mouse MCA occlusion model by the PIT method, the brain infarction volume ratios for groups administered 0.1 mg/kg or 1 mg/kg of anti-human TF antibody 3 hours after the end of light exposure (Graphs A, B and C).

### EMBODIMENT FOR CARRYING OUT THE INVENTION

The invention is described more fully below.

In the present invention, “TF inhibitor” is not subject to any particular limitation, provided it is a substance which inhibits the function of TF and exhibits an anticoagulation action. Exemplary TF inhibitors include anti-human TF antibodies, tissue factor pathway inhibitor (TFPI), inactivated blood coagulation factor VII (FVIIai), nematode anticoagulant protein (rNAPc2), and soluble TF variants (Kelly, R. F. et al.: Blood volume 9, page 3219-3227 (1997)). In the present invention, preferable TF inhibitor is an anti-human TF antibody.

As used herein, “inhibition of TF coagulation action” refers to inhibition of the blood coagulation factor IX and X activating effects due to the binding of TF to blood coagulation factor VII (formation of a TF/blood coagulation factor VII complex). Accordingly, “inhibition of TF coagulation action” encompasses inhibition of the binding of TF with blood coagulation factor VII (inhibition of the formation of TF/blood coagulation factor VII complex) and inhibition of the activation of blood coagulation factor IX or X.

As used herein, “anti-human TF antibody” may refer to any antibody which recognizes human TF, although an antibody which specifically recognizes human TF is preferred. The recognized human TF may be human TF in any state, such as human TF alone and human TF that has formed a complex. The anti-human TF antibody may be a monoclonal antibody or a polyclonal antibody, although a monoclonal antibody is preferred in that uniform antibody can be stably produced. Mouse antibodies, human antibodies, chimeric antibodies, humanized antibodies and the like may be suitably used as the anti-human TF antibody, although the use of a humanized anti-human TF antibody is preferred.

Any of the numerous known antibodies of this type that have already been reported (e.g., WO 99/51743, WO 88/07543, WO 96/40921, WO 98/40408, WO 01/70984, WO 03/037911, WO 03/93422) may be used as the anti-human TF antibody. Alternatively, because the tissue factor which serves as the antigen is already known (Ito, T. et al.: J. Biochem. Volume 114, page 691-696 (1993)), the anti-human TF antibody may be prepared by a method known to those skilled in the art, such as the following.

First, a gene sequence which codes for human TF is inserted into a known expression vector system, which is subsequently used to transform a suitable host cell. The target human TF protein can then be purified by a known method from the resulting host cell or a culture supernatant.

The purified human TF protein can then be used as a sensitizing antigen. Alternatively, a partial peptide of human TF may be used as the sensitizing antigen. The partial peptide may be obtained by chemical synthesis from the amino acid sequence of human TF.

No particular limitation is imposed on the epitope recognized by the anti-human TF antibody of the invention. That is, any epitope which is present on the human TF molecule may be recognized. Therefore, any fragment which includes an epitope present on the human TF molecule may be used as the antigen for preparing the anti-human TF antibody of the invention.

No particular limitation is imposed on the type of mammal which is sensitized with the above-described antigen, although it is preferable when selecting the type of mammal to take into account its compatibility with the parent cells used in cell fusion. Generally, use is made of a rodent (e.g., mouse, rat, hamster), or of a rabbit or monkey.

Immunization of the animal with the above antigen is carried out by a known immunization method. For example, a common method involves peritoneal or subcutaneous injection of the antigen into the mammal. Specifically, the antigen is diluted to the appropriate concentration and suspended in phosphate-buffered saline (PBS), physiological saline or the like. If desired, a suitable amount of an ordinary adjuvant, such as Freund's complete adjuvant, is mixed therewith and emulsified. The resulting preparation is administered several times to the mammal at intervals of from 4 to 21 days. Alternatively, a suitable carrier may be used at the time of antigen immunization.

After the mammal has been immunized in this way and it has been confirmed that the level of the desired antibody has risen in the blood serum, immune cells are collected from the mammal and furnished for cell fusion. Especially preferred immune cells include spleen cells.

Mammalian myeloma cells are used as the parent cells which are fused with the above-described immune cells. Various known cell lines are suitable as such myeloma cells, including P3 (P3x63Ag8.653) (J. Immunol. Volume 123, page 1548-1550 (1979)), P3x63Ag8U.1 (Current Topics in Microbiology and Immunology volume 81, page 1-7 (1978)), NS-1 (Kohler, G. and Milstein, C.: Eur. J. Immunol. Volume 6, page 511-519 (1976)), MPC-11 (Margulies, D. H. et al.: Cell volume 8, page 405-415 (1976)), SP2/0 (Shulman, M. et al.: Nature volume 276, page 269-270 (1978)), FO (dest. Groth, S. F. et al.: J. Immunol. Methods volume 35, page 1-21 (1980)), S194 (Trowbridge, I. S.: J. Exp. Med. Volume 148, page 313-323 (1978)) and R210 (Galfre, G. et al.: Nature volume 277, page 131-133 (1979)).

Cell fusion between the above immune cells and the myeloma cells may be carried out basically according to a known method, such as the method of Kohler and Milstein (Kohler, G. and Milstein, C.: Methods Enzymol. Volume 73, page 3-46 (1981)).

More specifically, such cell fusion is performed within a conventional nutrient medium in the presence of, for example, a cell fusion promoter. Examples of suitable fusion promoters include polyethylene glycol (PEG) and Sendai virus (HVJ). In addition, if desired, an auxiliary substance such as dimethylsulfoxide may be added and used to increase the fusion efficiency.

The relative proportions in which the immune cells and the myeloma cells are used may be set as desired. However, it is preferable for the number of immune cells to be from one to ten times relative to the number of myeloma cells. The culture medium used for such cell fusion may be, for example, the RPMI 1640 medium or the MEM medium, both of which are well-suited for growing the above-mentioned myeloma cell strains, or a common medium capable of being employed to cultivate this type of cell. In addition, concomitant use may be made of a serum supplement such as fetal calf serum (FCS).

Cell fusion is carried out by thoroughly mixing the above immune cells and myeloma cells in given amounts within the above-mentioned medium, adding a solution of PEG (e.g., of an average molecular weight of about 1,000 to about 6,000) that has been pre-warmed to about 37° C. to a concentration of typically 30 to 60% (w/v), and mixing, thereby forming the desired fused cells (hybridomas). Next, cell fusion chemicals and other substances undesirable to growth of the hybridomas are removed by repeatedly performing the successive operations of adding a suitable liquid medium, centrifugation, and removal of the supernatant.

The hybridomas obtained in this way are selected by cultivation in an ordinary selective medium, such as the HAT medium (a medium containing hypoxanthine, aminopterine and thymidine). Cultivation in this HAT medium is continued for a length of time (generally from several days to several weeks) sufficient to destroy all cells (non-fused cells) other than the target hybridomas. Next, screening of the hybridomas that produce the desired antibody and monocloning are carried out using a conventional limiting dilution method.

Apart from obtaining the above hybridomas by immunizing non-human animals with antigens, desired human antibodies having a TF-binding activity can be obtained by sensitizing human lymphocytes with TF in vitro, then fusing the sensitized lymphocytes with human-derived, permanently mitotic myeloma cells (see Japanese Patent Publication No. H 1-59878). Human antibodies to TF can also be obtained by administering TF as the antigen to a transgenic animal having the entire repertoire of human antibody genes so as to obtain anti-TF antibody-producing cells, immortalizing the cells, and collecting the antibody from the immortalized cells (see WO 94/25585, WO 93/12227, WO 92/03918, WO 94/02602).

Monoclonal antibody-producing hybridomas created in this way may be continuously cultivated in an ordinary medium, and may be stored for a long time in liquid nitrogen.

To obtain a monoclonal antibody from such hybridomas, use is typically made of, for example, a method in which the hybridomas are cultivated by an conventional technique and obtained as the culture supernatant thereof, or a method in which the hybridomas are administered to a mammal having compatibility therewith and thus induced to proliferate, then collected as peritoneal fluid. The former method is suitable for obtaining high-purity antibodies, whereas the latter method is suitable for producing a large amount of the antibody.

The monoclonal antibodies used in the present invention may be isolated from a phage antibody library (Clackson et al.: Nature volume 352, page 624-628 (1991); Marks et al.: J. Mol. Biol. volume 222, page 581-597 (1991)). The foregoing references from Clackson et al. and Marks et al. disclose, respectively, the isolation of mouse and human antibodies using phage libraries.

The monoclonal antibody used in the practice of the invention may be a recombinant monoclonal antibody obtained by cloning the antibody gene from the hybridoma, integrating a suitable vector, introducing the resulting product into a host, and production using genetic recombination technology (e.g., see Vandamme, A. M. et al.: Eur. J. Biochem. Volume 192, page 767-775 (1990)).

Specifically, mRNA coding for the variable (V) region of the anti-TF antibody is isolated from hybridomas which produce the anti-TF antibody. mRNA isolation is carried out by using a known technique such as guanidine ultracentrifugation (Chirgwin, J. M. et al.: Biochemistry volume 18, page 5294-5299 (1979)) or the AGPC method (Chomczynski, P. et al.: Anal. Biochem. Volume 162, page 156-159 (1987) to prepare the total RNA, and the target mRNA is prepared by using an mRNA Purification Kit (available from Pharmacia). Alternatively, mRNA may be prepared directly using the QuickPrep mRNA Purification Kit (available from Pharmacia).

The cDNA of the V region of the antibody is synthesized from the resulting mRNA using reverse transcriptase. Synthesis of the cDNA is carried out using, for example, the AMV Reverse Transcriptase First-strand cDNA Synthesis Kit (available from Seikagaku Corporation). Alternatively, cDNA synthesis and amplification may be carried out using, for example, the 5′-RACE method (Frohman, M. A. et al.: Proc. Natl. Acad. Sci. USA volume 85, page 8998-9002 (1988); Belyavsky, A. et al.: Nucleic Acids Res. Volume 17, page 2919-2932 (1989)) using the 5′-Ampli FINDER RACE Kit (available from Clontech) and the polymerase chain reaction (PCR).

The target DNA fragment is purified from the resulting PCR product, and ligated with vector DNA. This is then used to prepare a desired recombination vector by producing therefrom a recombinant vector, inserting the vector into, e.g., Escherichia coli, and colony selection. Next, the target DNA base sequence is confirmed by a known technique, such as the dideoxynucleotide chain termination method.

After obtaining DNA coding for the V region of the target anti-TF antibody, this DNA is integrated into an expression vector which contains DNA coding for the constant region (C region) of the desired antibody.

To produce the anti-human TF antibody used in the invention, the antibody gene is generally integrated into an expression vector for expression by an expression control region, such as under the control of an enhancer and a promoter. The host cells are then transformed by this expression vector, thereby expressing the antibody.

Expression of the antibody gene may be carried out either by integrating DNA coding for the heavy (H) chain and the light (L) chain of the antibody into separate expression vectors and cotransforming the host cell, or by integrating DNA coding for the H chain and the L chain into a single expression vector and transforming the host cell (see WO 94/11523).

It is possible to use not only the above-described host cells, but also transgenic animals in the production of recombinant antibodies. For example, the antibody gene may be prepared as a fusion gene by insertion on a gene which encodes a protein (e.g., goat β-casein) that is produced specifically in milk. DNA fragments containing the fusion gene obtained by insertion of the antibody gene are then injected into goat embryos, and the embryos are implanted in a female goat. The desired antibodies are obtained from milk produced by transgenic goats born to the goat in which the embryos were implanted, or by offspring of the transgenic goats. Suitable hormones may be used in the transgenic goats to increase the amount of milk containing the desired antibodies that is produced by the transgenic goats (Ebert, K. M., et al.: Bio/Technology 12, 699-702 (1994)).

The antibodies of the invention includes not only the above-described antibodies, but also modified antibodies that have been artificially modified for such purposes as to lower heteroantigenicity to humans, such as chimeric antibodies and humanized antibodies. These modified antibodies may be produced using known methods.

Chimeric antibodies may be obtained by ligating antibody V region-encoding DNA obtained as described above with, for example, human antibody C region-encoding DNA, integrating the resulting DNA into an expression vector, and introducing the vector into a host to produce antibody. Chimeric antibodies may be obtained using this known method.

Humanized antibodies are also referred to as “reshaped human antibodies.” These are obtained by grafting a complementarity determining region (CDR) of a non-human mammalian antibody, e.g., a mouse antibody, to the complementarity determining region of a human antibody. Common genetic recombination techniques for doing this are also known (see EP 125023, WO 96/02576).

Specifically, DNA sequences designed so that the CDR of a mouse antibody ligates with the framework region (FR) of a human antibody are synthesized by the PCR method using as the primers a plurality of oligonucleotides created so as to have portions which overlap with the end regions of both the CDR and the FR (see the method described in WO 98/13388).

A framework region in which the complementarity determining region forms a good antigen-bonding site is selected as the human antibody framework region which is ligated through the CDR. If necessary, framework region amino acids in the variable regions of the antibody may be substituted (Sato, K. et al.: Cancer Res. 53, 851-856 (1993)) so that the complementarity determining region of the reshaped human antibody forms a suitable antigen-bonding site.

The C region of a human antibody is generally used in the C regions of chimeric antibodies and humanized antibodies. For example, Cγ1, Cγ2, Cγ3 and Cγ4 may be used as the H chain, and C_{κ} and C_{λ} may be used as the L chain. The human antibody C region may be modified to improve the stability of the antibody and its production.

Because chimeric antibodies and humanized antibodies have a reduced antigenicity in the human body, they are thought to be useful for administration in humans, such as for therapeutic purposes.

The antibody used in the invention is not limited to the entire antibody molecule. So long as the antibody used in the invention recognizes TF, it may be an antibody fragment or a modified form thereof. Divalent antibodies and monovalent antibodies may also be used. Exemplary antibody fragments include Fab, F(ab′)2, Fv, Fab/c having a single Fab and a complete Fc, or a minibody, diabody or single-chain Fv (scFv) to which an H chain or an L chain Fv has been ligated with a suitable linker. Specific examples include those obtained by treating the antibody with an enzyme, such as papain or pepsin, to form antibody fragments, or constructing genes encoding for these antibody fragments, inserting the genes in an expression vector, then inducing expression in a suitable host cell (e.g., Co, M. S. et al.: J. Immunol. Volume 152, page 2968-2976 (1994); Better, M. and Horwitz, A. H.: Methods in Enzymology volume 178, page 476-496 (1989); Academic Press Inc., Plueckthum, A. and Skerra, A.: Methods in Enzymology volume 178, page 476-496 (1989); Academic Press Inc., Lamoyi, E.: Methods in Enzymology volume 121, page 652-663 (1989); Rousseaux, J. et al.: Methods in Enzymology volume 121, page 663-669 (1989); Bird, R. E. et al.: TIBTECH volume 9, page 132-137 (1991)).

scFv may be obtained by ligating the H chain V region of the antibody with the L chain V region. In this scFv, the H chain V region and the L chain V region are preferably ligated by means of linkers, preferably peptide linkers (Huston, J. S. et al.: Proc. Natl. Acad. Sci. U.S.A. volume 85, page 5879-5883 (1988)). The H chain V region and L chain V region in scFv may come from any of the antibodies mentioned in this specification. The peptide linker which ligates the V region, may be, for example, any single-chain peptide of about 5 to 20 amino acid residues.

DNA coding for scFv may be obtained by amplification with the PCR method using as the template a DNA fragment which, of the DNA which encodes the H chain or H chain V region of the antibody and the DNA which encodes the L chain or L chain V region, is DNA that encodes all of these sequences or a desired amino acid sequence thereof and using a primer pair that specifies both ends of the template DNA, followed by combination of DNA that codes for a portion of the peptide linker with primer pairs which specify the ligation of both ends of the DNA with the H chain and the L chain, respectively, and amplification thereof.

Once DNA encoding scFv is created, an expression vector containing such DNA and a host transformed with the expression vector may be obtained by conventional methods. Moreover, scFv may be obtained by a conventional technique using this host.

A minibody refers to an antigen-binding protein in which the VL and VH domains of a naturally occurring antibody are fused to the hinge region and CH3 domain of an immunoglobulin (for a minibody preparation method, see, for example, U.S. Pat. No. 5,837,821).

These antibody fragments may be produced in a host by acquiring and expressing the genes thereof in the same way as described above. The term “antibody” as used herein encompasses also these antibody fragments. Antibody modification products are exemplified by anti-TF antibodies that are bonded with various molecules, such as polyethylene glycol (PEG). The term “antibody” as used herein encompasses also such antibody modification products that are bonded with other substances. Such antibody modification products may be obtained by chemically modifying the antibody obtained. Methods for modifying antibodies are already established in the art.

The antibody used in the invention may be a bispecific antibody. The bispecific antibody may be a bispecific antibody having antigen binding sites which recognize different epitopes on the TF molecule, or a bispecific antibody on which one antigen binding site recognizes TF, and the other antigen binding site recognizes another substance. The bispecific antibody may be constructed by bonding together the HL pairs of two different antibodies, or may be obtained by fusing hybridomas that produce different monoclonal antibodies to create bispecific antibody-producing fused cells. In addition, bispecific antibodies may be created by genetic engineering techniques.

The antibody genes constructed as described above may be acquired through expression by a known method. In the case of mammalian cells, a commonly employed and useful promoter, the antibody gene to be expressed, and a poly A signal downstream from the 3′ side thereof may be functionally bonded and expressed. The promoter/enhancer is exemplified by a human cytomegalovirus immediate early promoter/enhancer.

Other examples of promoter/enhancers that may be used to express the antibody used in the present invention include promoter/enhancers of viruses such as retroviruses, polyomaviruses, adenoviruses and simian virus 40 (SV40); and promoter/enhancers derived from mammalian cells such as human elongation factor 1α (HEF1α).

Gene expression may readily be carried out by the method of Mulligan et al. (Nature volume 277, page 108 (1979)) when the SV40 promoter/enhancer is used, and by the method of Mizushima et al. (Nucleic Acids Res. Volme 18, page 5322 (1990)) when the HEF1α promoter/enhancer is used.

In the case of E. coli, a useful, commonly used promoter, a signal sequence for antibody secretion, and the antibody gene to be expressed may be functionally bonded, and the gene expressed. The promoter is exemplified by the lacZ promoter and the araB promoter. Gene expression may be carried by the method of Ward et al. (Nature volume 341, page 544-546 (1998); FASEB J. volume 6, page 2422-2427 (1992)) when the lacz promoter is used, and by the method of Better et al. (Science volume 240, page 1041-1043 (1988)) when the araB promoter is used.

The signal sequence used for antibody secretion may be the PelB signal sequence (Lei, S. P. et al.: Bacteriol. Volume 169, page 4379 (1987)) when production is induced in E. coli periplasm. After separating the antibody produced in the periplasm, the structure of the antibody is refolded for use.

Material from SV40, polyomaviruses, adenoviruses or bovine papillomaviruses (BPV) may be used as the replication origin. In addition, to amplify the number of gene copies in the host cell system, the expression vector may include any of the following as the selected marker: an aminoglycoside transferase (APH) gene, a thymidine kinase (TK) gene, an E. coli xanthine guanine phosphoribosyl transferase (Ecogpt) gene, a dihydrofolate reductase (dhfr) gene, etc.

Any expression system, such as a eukaryotic cell or a prokaryotic cell system, may be used to produce the antibody used in the invention. Examples of eukaryotic cells include established mammalian cell line, insect cell line, mold cells and yeast cells. Examples of prokaryotic cells include bacterial cells such as E. coli cells.

The antibodies used in the invention are preferably expressed in mammalian cells, such as CHO, COS, myeloma, BHK, Vero or HeLa cells.

Next, the transformed host cells are cultured in vitro or in vivo to produce the target antibodies. Cultivation of the host cells is carried out according to a known method. For example, the medium used may be DMEM, MEM, RPMI 1640 or IMDM. A serum supplement such as fetal calf serum (FCS) may be used together.

The antibodies expressed and produced as described above may be isolated and purified to uniformity from the cells or host animal. Isolation and purification of the antibodies used in the invention may be carried out using an affinity column. Examples columns as suitable protein A columns include Hyper D, POROS and Sepharose F.F. (available from Pharmacia). Any other method of separation and purification commonly used for proteins may be used without particular limitation. For example, the antibodies may be isolated and purified by suitably using and combining chromatography columns other than the above-described affinity columns, filters, ultrafiltration, salting out, and dialysis (Antibodies: A Laboratory Manual, Ed. Harlow, David Lane (Cold Spring Harbor Laboratory, 1988)).,

A known method may be used to measure the antigen binding activity of the antibodies (Antibodies: A Laboratory Manual, Ed. Harlow, David Lane (Cold Spring Harbor Laboratory, 1988)).

The method employed to measure the antigen binding activity of the anti-TF antibodies used in the invention may be ELISA (enzyme-linked immunosorbent assay), EIA (enzyme immunoassay), RIA (radioimmunoassay) or the fluorescent antibody technique. For example, when an enzyme immunoassay is used, an anti-TF antibody-containing specimen, such as anti-TF antibody-producing cell culture supernatant or purified antibody, is added to a TF-coated plate, after which a secondary antibody labeled with an enzyme such as alkali phosphatase is added, and then the plate is incubated and washed. By then adding an enzyme substrate such as p-nitrophenylphosphoric acid and measuring the absorbance, the antigen binding activity can be evaluated.

In the practice of the invention, “thromboembolic ischemic disease” refers to ischemic diseases that are caused or partly caused by a thrombus or an embolism. Specific examples of such ischemic diseases include arterial thromboses, such as infarctions (e.g., brain infarction, myocardial infarction), TIA, angina pectoris and chromic arterial obstruction; and venous thromboses, such as deep-vein thrombosis of the lower limbs, pulmonary embolism and cerebral sinus thrombosis.

In the practice of the invention, the thromboembolic ischemic disease may be exemplified by an infarction. The site of the infarction is not subject to any particular limitation. The infarction may be one that occurs at any site, such as in the brain or the heart.

Infarctions in this specification include ischemic infarctions in, for example, the brain, heart, lungs, kidneys and spleen. In the practice of the invention, an infarction in the brain (brain infarction) is suitable. Brain infarctions include clinical entities such as atherothrombotic brain infarctions, lacunar infarctions and cardiogenic brain embolism.

In the practice of the invention, “following onset of ischemic disease” signifies after an ischemic disease condition caused by vascular stenosis or occlusion has been confirmed. This condition may be severe or mild, and encompasses both conditions which intensify by gradual progression and conditions which abruptly reach completion. Diagnosis and judgment of onset and the stage of onset is generally carried out by a physician or the equivalent. The period of administration of the invention pharmaceutical composition is not subject to any particular limitation, so long as it is administered following the onset of ischemic disease. For example, the pharmaceutical composition may be given in the acute or hyperacute stage following the onset of ischemic disease. Specifically, the pharmaceutical composition may be administered within 48 hours of onset, preferably within 24 hours of onset, more preferably within 6 hours of onset, and most preferably within 3 hours of onset. The inventive pharmaceutical composition may also be used for chronic treatment.

As used herein, the phrase “following onset of an infarction” encompasses both “following confirmation of the presence of an infarction” and “following a determination that there is a high probability of the presence of an infarction or that an infarction is present.” Also, the phrase “following thrombus or embolism formation” encompasses both “following confirmation of the presence of a thrombus or an embolism” and “following a determination that there is a high probability of the presence of a thrombus or embolism or that a thrombus or embolism is present.” The presence of an infarction, thrombus or embolism can generally be confirmed by, for example, computerized tomography (CT), magnetic resonance imaging (MRI), magnetic resonance angiography (MRA) or angiography. The phrase “suppressing enlargement of an infarction” means to suppress enlargement of the infarction volume and/or the infarction surface area.

As used herein, the term “primary thrombus” refers to a thrombus which initially formed or was confirmed at the site of the lesion or in the vicinity thereof.

As used herein, the term “secondary thrombus” denotes a thrombus which forms after formation of the primary thrombus. Specific examples include a second thrombus, a third thrombus and a fourth thrombus (Norio Tanahashi: No to Junkan (brain and circulation) volume 4, page 319-325 (1997)). A thrombus which forms after formation of the primary thrombus is called the “second thrombus”, and thrombi which form after formation of the second thrombus are referred to as, in order, the “third thrombus”, “fourth thrombus”, “fifth thrombus”, etc. Secondary thrombi include thrombi which form at a different site in the presence of the primary thrombus, and also thrombi which form at the same or different sites following resolution of the primary thrombus. The inhibition of secondary thrombus formation refers herein to completely inhibiting the formation of secondary thrombi, reducing the amount of secondary thrombus formation, retarding the period of secondary thrombus formation, or reducing the size of secondary thrombi that will form or have formed.

“Suppressing a reduction in blood flow” refers herein to suppressing a reduction in the amount of blood flowing through the blood vessels, and typically refers herein to suppressing the amount of blood flowing through blood vessels at the site of the lesion. Measurement of the blood flow may be carried out by a method known to those skilled in the art.

When the pharmaceutical composition of the invention includes as an active ingredient an antibody which binds with TF, the pharmaceutical composition may be administered parenterally, either systemically or topically. The mode of administration may be suitably selected, according to the age of the patient and the symptoms, from among, for example, intravenous injection such as an intravenous drip, intramuscular injection, intraperitoneal injection and subcutaneous injection. The effective dose per kilogram of body weight per administration is selected in a range of from 0.001 to 100 mg, preferably from 0.01 to 10 mg, and most preferably from 0.1 to 1 mg.

When the pharmaceutical composition of the invention includes as an active ingredient an antibody which binds with TF, the pharmaceutical composition may also include pharmaceutically acceptable carriers and excipients, depending on the route of administration. Such carriers and excipients are exemplified by water, pharmaceutically acceptable organic solvents, collagen, polyvinyl alcohol, polyvinylpyrrolidone, carboxyvinyl polymer, carboxymethylcellulose sodium salt, sodium polyacrylate, sodium alginate, water-soluble dextran, sodium carboxymethyl starch, pectin, methyl cellulose, ethyl cellulose, xanthan gum, gum arabic, casein, gelatin, agar, diglycerin, glycerin, propylene glycol, polyethylene glycol, petrolatum, paraffin, stearyl alcohol, stearic acid, human serum albumin (HAS), mannitol, furbitol, lactose, and surfactants acceptable as pharmaceutical excipients. The excipients that are used may be suitably selected individually or in combination from among those mentioned above according to the dosage form of the invention, but are not limited only to these.

The pharmaceutical composition of the invention may be used in combination drug therapy by administration at the same time as, consecutive to, or separate from, one or more other drug, such as an antiplatelet drug, a thrombolytic drug or an anticoagulant. In combination drug therapy, preferred antiplatelet drugs include aspirin, ticlopidine hydrochloride and ozagrel sodium, preferred thrombolytic drugs include urokinase and t-PA, and preferred anticoagulants include warfarin, heparin and argatroban. Moreover, the pharmaceutical compositions of the invention may be used in combination with surgical therapy.

### EXAMPLES

Examples are given below to more fully illustrate the present invention, but are not intended to limit the scope of the invention. Based on the descriptions of the invention, various changes and modifications will be apparent to those skilled in the art and shall be understood as being embraced by the invention.

1) Test Specimens and Experimental Animals

The test specimens used in the examples were prepared by the following methods or procured.

Anti-human TF antibody was prepared by the method described in WO 99/51743 (the heavy chains were the humanized H chain version i described in WO 99/51743, and the light chains were the humanized L chain version b2 described in WO 99/51743). The anti-human TF antibody was prepared as a solution (15.4 mg/mL) in a solvent (20 mmol/L sodium acetate, 150 mmol/L NaCl, pH 6.0), and stored by freezing (temperature setting: −80° C.) until the time of use.

A buffer (20 mmol/L sodium acetate, 150 mmol/L NaCl, pH 6.0) for diluting the anti-human TF antibody was prepared, then stored by refrigeration (temperature setting: 4° C.) until the time of use. The anti-human TF antibody was diluted with an anti-human TF antibody-diluting buffer in such a way as to set the doses for the respective samples to 0.01 mg/5 mL/kg, 0.03 mg/5 mL/kg, 0.1 mg/5 mL/kg, 1 mg/5 mL/kg and 10 mg/5 mL/kg. Samples were prepared on the day of administration, and stored at room temperature following preparation.

Because the anti-human TF antibody exhibits cross-reactivity only with primate antigens, hTF-KI mice (see WO 02/94016) (line 76) in which the human TF was expressed were used in the drug evaluation tests in the examples. Male mice 10 to 19 weeks of age with body weights of 21.6 to 35.7 g (at time of MCA occlusion experiment) were randomly divided into groups. The habituation period was set at 5 or more days, and the state of health was assessed by visual observation of the general condition of the animals.

2) Test Method

(2-1) Creation of Mouse MCA Occlusion Model by Photochemically Induced Thrombosis (PIT)

The mice were anesthetized by isoflurane inhalation (background anesthesia; oxygen, 30%; nitrous oxide, 70%). The anesthetized animals were immobilized in a supine position while breathing naturally. The rectal temperature was held near 37° C. with a thermal pad and an incandescent lamp, and the temperature just prior to light irradiation was recorded. Following disinfection of the neck region with Isodine (Meiji Seika Kaisha, Ltd.), an incision was made in the skin of the neck and the left jugular vein was detached. A PE-10 catheter was inserted into the jugular vein and secured with suture thread. Next, the position of the animal was changed so that the left side faced up. After coating the left side of the head with Isodine, an incision was made in the skin midway between the left external ear and the left external canthus so as to expose the skull and the temporal muscle. An incision was made in the temporal muscle, and the temporal muscle was separated from the skull using microtweezers. The middle cerebral artery (MCA), which appears transparent from above the skull, was visually confirmed at this point. An electric drill was placed against the skull and a hole about 2 to 3 mm in diameter was drilled in the skull so as to leave a thin layer of bone intact, following which the thin layer of bone was removed with microtweezers. Using a micromanipulator, a light-irradiating probe (A5355MOD, Hamamatsu Photonics K. K.) was placed over the MCA visible at the position of the hole in the bone, and xenon green light (540 nm) was irradiated for 10 minutes at an intensity of 3,000 lux (0.4 W/cm²) using a light source for thrombus model creation (L4887, Hamamatsu Photonics K. K.). Concurrent with the start of irradiation, a rose bengal solution (dosage, 20 mg/mL/kg; the solvent was physiological saline) was administered through the jugular vein catheter for about 1 minute. Following the end of light irradiation, the incisions were closed, and the animal was promptly brought out of anesthesia. The animal was then kept for a fixed period of time (24 hours) until removal of the brain.

(2-2) Measurement of Blood Flow in Brain

At the time of mouse MCA occlusion model creation by the PIT method, a laser Doppler tissue blood flowmeter probe (ST-N, Omega Flow) was placed at a parietal site directly above and distal to the light irradiation probe and, using a non-contact type laser Doppler tissue blood flowmeter (FLO-N1, Omega Flow), the brain tissue blood flow rate was continuously measured from about 5 minutes prior to and during light irradiation (10 minutes). The time from the start of light irradiation until a certain standard reduction in blood flow (time to occlusion, abbreviated below as “TTO”) and the residual blood flow at certain standard times were determined as indicators of the decrease in the brain blood flow. The “certain standard” blood flow reductions were as follows:
1. lowest blood flow
2. stable reduction in blood flow of at least one minute (plateau).

(2-3) Quantifying the Brain Infarctions

Twenty-four hours after MCA occlusion, the mice were anesthetized by isoflurane inhalation (background anesthesia: oxygen, 30%; nitrous oxide, 70%) and the brains promptly removed by decapitation. The extracted brains were sectioned rostrad from the boundary between the cerebrum and the cerebellum at a thickness of 1 mm, creating six consecutive coronal sections of the cerebrum which were used to quantify the brain infarction. The brain sections were immersed in physiological saline containing 2% (w/v) 2,3,5-triphenyltetrazolium chloride (abbreviated below as “TTC”) held warm in a 37° C. gas-phase incubator, and stained by being kept warm for 30 minutes in the same incubator. The sections were then fixed by immersion in a 10% neutral buffered formalin solution. The rostral side of each section was photographed, and the TTC stained region (normal tissue region) and unstained region (infarcted region) were marked off on the photograph. The infarcted region was divided into two parts: the cerebral cortex region (cortex) and regions other than the cerebral cortex (subcortex), and quantification was carried out. Totals for the cortex regions collectively and for the subcortex regions collectively were also determined and analyzed. The infarction volume was determined by multiplying the surface area of the infarction by the thickness of the section (1 mm) to calculate the infarction volume for each section, then adding together the values obtained for the six sections. Taking into account the influence of infarction enlargement due to brain edema, corrections were carried out based on the ratio between the contralateral cerebral surface area and the cerebral surface area on the affected side. The infarction surface area and volume results were all computed as corrected values (see Stroke 24, 117-121 (1993)). Last of all, the infarction volume as a proportion of the cerebral hemisphere on the affected side was computed.

3) Evaluation of Drug Efficacy of Anti-Human TF Antibody

(3-1) Structure of Drug Efficacy Test

The brain infarction-suppressing effect of a single intravenous administration of anti-human TF antibody was investigated using the mouse MCA occlusion model by the PIT method. In this test, the following three experiments were carried out in which the timing and dose of anti-human TF antibody administration was varied.

Experiment 1: Administered Shortly After End of Light Irradiation

Control group

0.1 mg/kg Anti-human TF antibody group

1 mg/kg Anti-human TF antibody group

### Experiment 2: Administered Shortly After End of Light Irradiation

Control group

0.01 mg/kg Anti-human TF antibody group

0.03 mg/kg Anti-human TF antibody group

0.1 mg/kg Anti-human TF antibody group

### Experiment 3: Administered 3 Hours After End of Light Irradiation

Control group

0.1 mg/kg Anti-human TF antibody group

1 mg/kg Anti-human TF antibody group

Administration of the anti-human TF antibody solution (anti-human TF antibody group) or the buffer (control group) used for diluting the anti-human TF antibody was carried out as a single dose following the end of light irradiation, and by the same route as administration of the rose bengal solution; i.e., from a catheter placed within the jugular vein. Administration was carried out with the creators of the MCA occlusion model blinded to the composition of the groups. Moreover, equal and random assignment of the respective groups was carried out each day of the experiment. The efficacy of the anti-human TF antibody was evaluated using the above-described brain infarction volume as the indicator.

4) Statistical Analysis

In the-described above anti-human TF antibody efficacy evaluation test, the drug efficacy was evaluated by statistical analysis, and Dunnett's test was used to compare the infarction volumes between the control group and the anti-human TF antibody groups. In addition, using the reduction in blood flow within the brain as the indicator, the residual blood flow and TTO for the control group and the anti-human TF antibody groups were compared by Dunnett's test. Comparisons between the control group and the anti-human TF antibody groups in the body weights of the mice and the mouse rectal temperature at the time of surgery were likewise carried out by Dunnett's test. The results were all indicated as the average value±standard error. In two-sided testing, p<0.05 was regarded as statistically significant. The WEB edition SAS (Ver. 6.12) was used as the statistical software.

5) Results

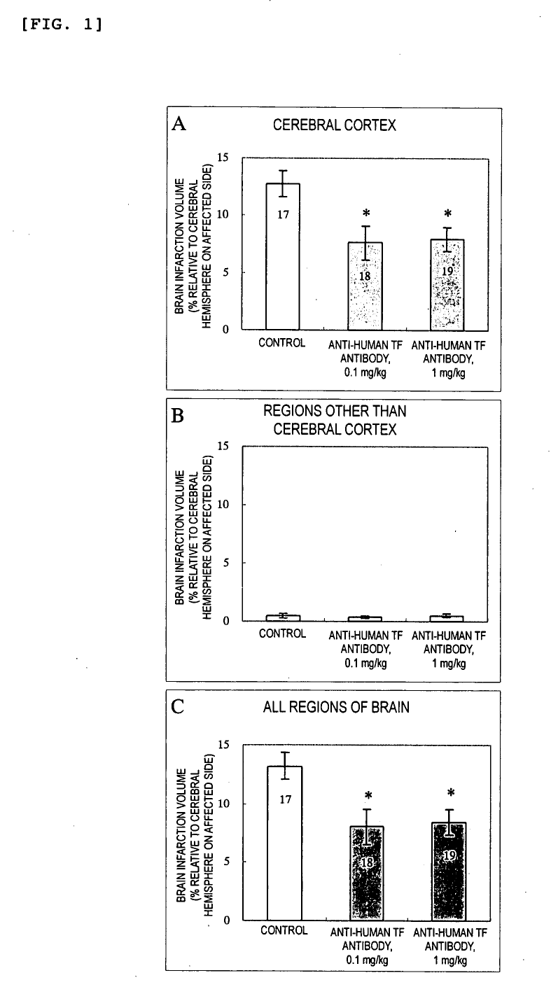

Infarction volumes are shown in FIGS. 1 to 3 as a ratio (%) with respect to the cerebral hemisphere on the affected side. In FIGS. 1 to 3, in addition to quantifying the infarcted region for all regions of the brain (total), quantification results are also shown separately for the cerebral cortex and for other regions of the brain (subcortex).

Experiment 1: Administration Shortly After End of Light Irradiation

Significant brain infarction inhibiting effects were observed in the cortex and the total brain with the administration of 0.1 mg/kg of anti-human TF antibody and 1 mg/kg of anti-human TF antibody (see FIG. 1).

Experiment 2: Administration Shortly After End of Light Irradiation (Study of Low-Dose Effects)

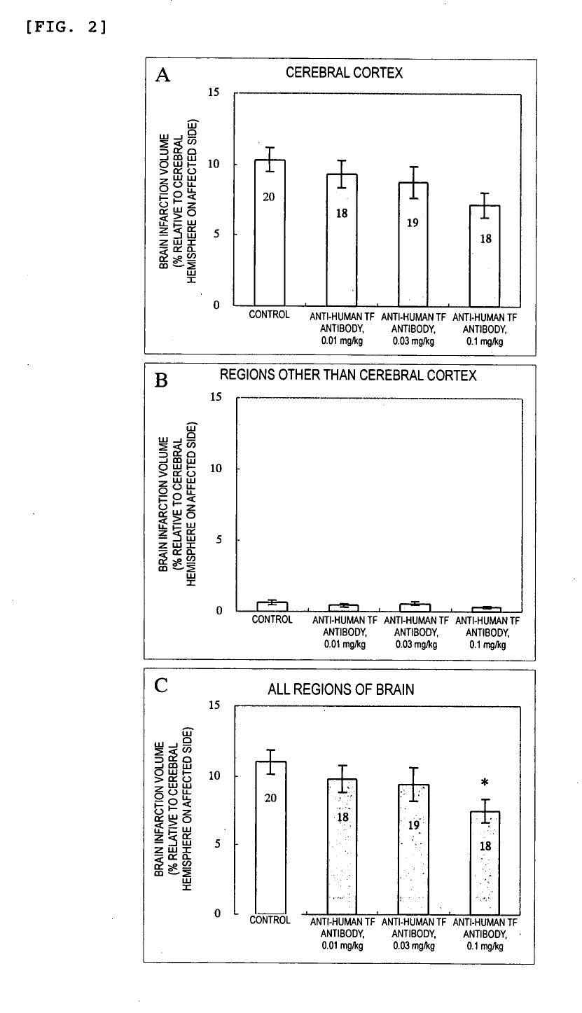

Significant brain infarction inhibiting effects were observed in the total brain with the administration of 0.1 mg/kg of the anti-human TF antibody (see FIG. 2).

Experiment 3: Administration 3 Hours After End of Light Irradiation

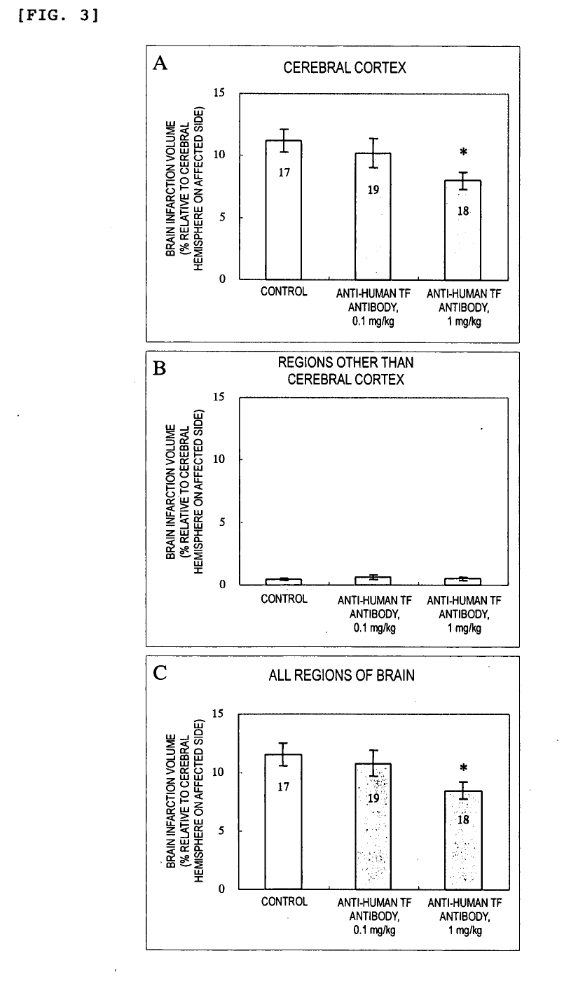

Significant brain infarction inhibiting effects were observed in the cortex and the total brain with the administration of 1 mg/kg of anti-human TF antibody (see FIG. 3).

Mouse weight, rectal temperature and brain blood flow measurements in Experiments 1 to 3 are shown respectively in Tables 1 to 3.

TABLE 1

| | Rectal | Residual blood | | | |
|---|---|---|---|---|---|
| Weight | temp. | flow (%) | | TTO (min) | |
| (g) | (° C.) | lowest | plateau | lowest | plateau |

Control

| | | | | | | |
|---|---|---|---|---|---|---|
| Mean | 28.6 | 36.8 | 43.1 | 62.3 | 4.4 | 6.4 |
| Standard error | 0.6 | 0.2 | 4.0 | 6.4 | 0.3 | 0.5 |

Anti-human TF antibody: 0.1 mg/kg

| | | | | | | |
|---|---|---|---|---|---|---|
| Mean | 27.4 | 36.7 | 34.8 | 50.4 | 4.3 | 6.6 |
| Standard error | 0.6 | 0.2 | 4.4 | 7.0 | 0.4 | 0.5 |

Anti-human TF antibody: 1 mg/kg

| | | | | | | |
|---|---|---|---|---|---|---|
| Mean | 28.9 | 36.8 | 48.9 | 67.5 | 4.8 | 6.6 |
| Standard error | 0.8 | 0.1 | 5.2 | 7.7 | 0.6 | 0.5 |

TABLE 2

| | Rectal | Residual blood | | | |
|---|---|---|---|---|---|
| Weight | temp. | flow (%) | | TTO (min) | |
| (g) | (° C.) | lowest | plateau | lowest | plateau |

Control

| | | | | | | |
|---|---|---|---|---|---|---|
| Mean | 28.7 | 37.1 | 42.5 | 60.8 | 3.8 | 4.9 |
| Standard error | 0.6 | 0.1 | 3.5 | 6.6 | 0.4 | 0.5 |

Anti-human TF antibody: 0.01 mg/kg

| | | | | | | |
|---|---|---|---|---|---|---|
| Mean | 28.6 | 36.9 | 40.0 | 56.4 | 4.0 | 5.3 |
| Standard error | 0.7 | 0.1 | 3.7 | 5.7 | 0.4 | 0.4 |

Anti-human TF antibody: 0.03 mg/kg

| | | | | | | |
|---|---|---|---|---|---|---|
| Mean | 28.3 | 37.0 | 45.7 | 65.6 | 4.1 | 5.5 |
| Standard error | 0.5 | 0.2 | 5.5 | 8.1 | 0.4 | 0.5 |

Anti-human TF antibody: 1 mg/kg

| | | | | | | |
|---|---|---|---|---|---|---|
| Mean | 29.0 | 37.0 | 44.3 | 55.6 | 5.2 | 5.8 |
| Standard error | 0.8 | 0.1 | 6.0 | 6.9 | 0.6 | 0.6 |

TABLE 3

| | Rectal | Residual blood | | | |
|---|---|---|---|---|---|
| Weight | temp. | flow (%) | | TTO (min) | |
| (g) | (° C.) | lowest | plateau | lowest | plateau |

Control

| | | | | | | |
|---|---|---|---|---|---|---|
| Mean | 30.0 | 37.2 | 37.9 | 48.9 | 3.6 | 4.6 |
| Standard error | 0.7 | 0.2 | 3.4 | 6.3 | 0.3 | 0.5 |

Anti-human TF antibody: 0.1 mg/kg

| | | | | | | |
|---|---|---|---|---|---|---|
| Mean | 30.6 | 36.7 | 36.7 | 47.6 | 4.2 | 5.1 |
| Standard error | 0.6 | 0.2 | 3.0 | 4.5 | 0.4 | 0.4 |

Anti-human TF antibody: 1 mg/kg

| | | | | | | |
|---|---|---|---|---|---|---|
| Mean | 30.3 | 37.1 | 34.8 | 51.1 | 3.4 | 4.4 |
| Standard error | 0.7 | 0.2 | 1.9 | 5.2 | 0.2 | 0.2 |

In a mouse MCA occlusion model using the PIT method, thrombus formation (i.e., formation of a primary thrombus) occurred within the MCA during 10-minute light irradiation, whereupon blood flow in the brain rapidly decreased, leading to brain ischemia (see Tables 1 to 3). Throughout Experiments 1 to 3, significant differences in the rectal temperature at the time of surgery, residual blood flow and TTO did not appear between any of the groups. Therefore, the reduction in blood flow within the brain due to primary thrombus was about the same in the control group and the anti-human TF antibody groups, allowing the efficacy of anti-human TF antibody on brain infarction formation to be demonstrated under the same brain ischemia conditions.

It is apparent from the results of Experiments 1 to 3 that the anti-human TF antibody exhibited brain infarction suppressing effects even when administered following the end of light irradiation (i.e., after formation of the primary thrombus). In particular, because a brain infarction suppressing effect was observed with administration 3 hours after the end of light irradiation, it is apparent that anti-human TF antibody has a broad therapeutic time window (TTW), indicating that enlargement of a brain infarction can be suppressed even when administration is carried out following onset of the infarction.

Because anti-human TF antibody has a TF-inhibiting action, the mechanism by which a brain infarction suppressing effect arises is thought to involve suppression by the anti-human TF antibody of thrombus formation in which TF takes part, resulting in the suppression of the thrombus-induced reduction in brain blood flow. In this example, because the anti-human TF antibody was administered after primary thrombus formation, the thrombi whose formation was suppressed by anti-human TF antibody were, first of all, secondary thrombi which form at different sites in the presence of the primary thrombus. Secondly, it is known that when a primary thrombus that has been formed by the PIT method is partially resolved, reperfusion occurs, after which the process by which thrombus formation occurs is repeated at the same site. Anti-human TF antibody appeared to suppress the secondary thrombi which form at the same site as the primary thrombus.

This example relates to the efficacy of the inventive pharmaceutical composition against brain infarctions. However, the results of this example suggest that administering the inventive pharmaceutical composition will provide infarction-suppressing effects in other organs as well, such as the heart and lungs, by mechanisms similar to that seen here.

Moreover, not only in infarctions, but in all ischemic diseases that are caused at least in part by thrombosis or embolism, the formation of secondary thrombi is believed to cause a progressive reduction in blood flow and play a role in enlarging the pathology. Thus, the results of this example suggest that administering the inventive pharmaceutical composition following onset will provide a therapeutic effect on thrombus and embolism-induced ischemic diseases in general.

1. A pharmaceutical composition comprising a tissue factor inhibitor, which is administered following onset of thromboembolic ischemic disease.

2. The pharmaceutical composition according to claim 1, for use in treating thromboembolic ischemic disease.

3. The pharmaceutical composition according to claim 1, wherein the thromboembolic ischemic disease is an infarction.

4. The pharmaceutical composition according to claim 3, wherein the infarction is a brain infarction.

5. The pharmaceutical composition according to claim 1, wherein the tissue factor inhibitor is an antibody which binds to tissue factor.

6. A pharmaceutical composition comprising a tissue factor inhibitor, which is administered following thrombus or embolism formation.

7. The pharmaceutical composition according to claim 6, for use in treating thromboembolic ischemic disease.

8. The pharmaceutical composition according to claim 7, wherein the thromboembolic ischemic disease is an infarction.

9. The pharmaceutical composition according to claim 8, wherein the infarction is a brain infarction.

10. The pharmaceutical composition according to claim 6, wherein the tissue factor inhibitor is an antibody which binds to tissue factor.

11. A secondary thrombus formation inhibitor comprising a tissue factor inhibitor, which is administered following primary thrombus formation.

12. The secondary thrombus formation inhibitor according to claim 11, wherein the tissue factor inhibitor is an antibody which binds to tissue factor.

13. A blood flow reduction inhibitor comprising a tissue factor inhibitor to be administered following thrombus formation or embolism formation.

14. The blood flow reduction inhibitor according to claim 13, wherein the tissue factor inhibitor is an antibody which binds to tissue factor.

15. A method of suppressing secondary thrombus formation by inhibiting tissue factor function following formation of a primary thrombus.

16. A method of suppressing enlargement of an infarction by inhibiting tissue factor function following onset of the infarction.

17. A method of suppressing a reduction in blood flow by inhibiting tissue factor function following thrombus formation or embolism formation.
